(19) United States
(12) Patent Application Publication
(10) Pub. No.: US 2023/0238145 A1
(43) Pub. Date: Jul. 27, 2023

(54) HEALTH SCORE AND PREDICTIVE ANALYSIS

(71) Applicant: OLA MD LLC, WESLEY CHAPEL, FL (US)

(72) Inventor: Alpesh Patel, Wimauma, FL (US)

(21) Appl. No.: 18/101,143

(22) Filed: Jan. 25, 2023

Related U.S. Application Data
(60) Provisional application No. 63/302,795, filed on Jan. 25, 2022.

(51) Int. Cl. G16H 50/30; G16H 10/60

(52) U.S. Cl. CPC G16H 50/30; G16H 10/60

(57) ABSTRACT

A system for determining a patient health score and predicting a likelihood of a future medical condition comprises a patient device and a computer data processing system in communication with the patient device. The computer data processing system is configured to: receive, from the patient device, a set of patient records associated with a patient; store and analyze the set of patient records; generate and assign at least one score to the set of patient records based on the analysis of the set of patient records, each score is indicative of the likelihood of a future medical condition; generate at least one recommendation for improvement so that the patient can avoid the future medical condition; and transmit the at least one recommendation to the patient device.

### CROSS-REFERENCE TO RELATED APPLICATION(S)

This application claims priority to U.S. Provisional Patent Application No. 63/302,795, entitled HEALTH SCORE AND PREDICTIVE ANALYSIS, filed on Jan. 25, 2022, the entirety of which is incorporated herein by reference.

### FIELD OF THE INVENTION

The present invention relates to the field of telehealth, and more specifically, to systems and methods for determining a patient health score and conducting a predictive analysis of potential health conditions based on the patient health score.

### BACKGROUND OF THE INVENTION

Healthcare often works in a silo, with limited information on a patient's overall health due to time restrictions and limitations in training. A patient's doctor does not always talk to the patient's primary care doctor, mental health professional, or their dietician, and rarely do they talk to allied health professionals like personal fitness trainers to get a full grasp on a patients whole health needs. However, many conditions that a patient may face are often interrelated to other conditions and treatments. Therefore, a patient and their providers would benefit greatly from visibility of the complete health and wellness of the patient. Unfortunately, patients are frequently unaware of the interrelated issues often associated with co-morbid medical conditions and without specific intervention, result in developing increasingly severe medical conditions.

Existing healthcare systems do not enable healthcare providers to be proactive in recommending treatments to their patients that expand past their specialty. Existing systems often require a patient visit in order for the healthcare provider to examine the patient's medical history and recommend improvements or treatments in real-time based on the patients' medical history and current conditions. However, these systems do not allow healthcare providers the opportunity to receive all of the patient's healthcare information and conduct a predictive analysis so that the provider can recommend treatments or improvements so that the patient can lessen the likelihood that they experience a future medical condition. This has resulted in a healthcare system intended to provide medical condition focused intervention based care, instead of prevention focused based care.

### SUMMARY OF THE INVENTION

Embodiments of the present invention address deficiencies of the art in respect to telehealth and provide a novel and non-obvious apparatus, system, computer program product and method to determine a patient's health score and predictive analysis regarding the same. In an embodiment of the invention, a patient's medical claim records are collected and analyzed and a medical claims score is assigned. The patient's prescription records are collected and analyzed and a prescription score is assigned. The patient's diet records are collected and analyzed and a diet score is assigned. The patient's lifestyle records are collected and analyzed and a lifestyle score is assigned. The patient's lab results records are collected and analyzed and a lab results score is assigned. The patient's mental health records are collected and analyzed and a mental health score is assigned. The weightage for each of the medical claim records score, prescription score, diet score, lifestyle score, lab results score and mental health score is determined to calculate a total weighted health score. The total weighted health score is then displayed to the patient.

According to one or more embodiments of the invention, a method of determining a patient health score for predicting a likelihood of a future medical condition, comprises the steps of: receiving, from a patient, a set of patient records associated with the patient, the set of patient records is received by a computer data processing system maintained by an associated healthcare provider; storing, at the computer data processing system, the set of patient records; analyzing, at the computer data processing system, the set of patient records; generating and assigning, at the computer data processing system, at least one score to the set of patient records based on the analysis of the records, each score is indicative of the likelihood of a future medical condition; generating, at the computer data processing system, at least one recommendation for improvement to the patient so that the patient can avoid the future medical condition; and transmitting the at least one recommendation from the computer data processing system to a patient device.

In one aspect, the set of patient records includes at least one of medical claims records, prescription records, diet records, lifestyle records, lab result records, and mental health records.

In another aspect, the medical claims records, prescription records, diet records, lifestyle records, lab result records, and mental health records are each assigned a respective score.

In another aspect, the method further comprises: determining, via a health score logic of the computer data processing system, a weighting for each of the respective scores; and applying the weighting to each respective score to calculate a weighted score.

In another aspect, the method further comprises: determining a total weighted health score based on a sum of each weighted score; and providing the total weighted health score to the patient.

In another aspect, the computer data processing system has a Predictive Analysis Logic and the at least one recommendation for improvement is determined by the Predictive Analysis Logic.

In another aspect, the at least one recommendation from the computer data processing system to a patient device is transmitted via at least one of a wired and wireless connection to the patient device.

In another aspect, the method further comprises routinely updating the at least one score based on whether the patient follows the recommendation for improvement.

According to one or more embodiments, a system for determining a patient health score for predicting a likelihood of a future medical condition comprises a patient device and a computer data processing system in communication with the patient device. The computer data processing system is configured to: receive, from the patient device, a set of patient records associated with a patient; store and analyze the set of patient records; generate and assign at least one score to the set of patient records based on the analysis of the set of patient records, each score is indicative of the likelihood of a future medical condition; generate at least one recommendation for improvement so that the patient can avoid the future medical condition; and transmit the at least one recommendation to the patient device.

In one aspect, the set of patient records includes at least one of medical claims records, prescription records, diet records, lifestyle records, lab result records, and mental health records.

In another aspect, the medical claims records, prescription records, diet records, lifestyle records, lab result records, and mental health records are each assigned a respective score.

In another aspect, the computer data processing system is further configured to determine a weighting for each of the respective scores and apply each weighting to each respective score to calculate a weighted score.

In another aspect, the computer data processing system is further configured to determine a total weighted health score based on a sum of each weighted score and transmit the total weighted health score to the patient device.

In another aspect, the computer data processing system is configured to at least periodically update the total weighted health score based on whether the patient follows the recommended improvements.

In another aspect, the computer data processing system is operated by one or more healthcare providers.

In another aspect, the computer data processing system is in communication with the patient device by at least one of a wired and wireless connection.

In another aspect, the computer data processing system is in communication with a plurality of patient devices.

In another aspect, the computer data processing system is configured to receive a set of patient records from each patient device.

In another aspect, after the computer data processing system transmits the recommendation to the patient device, the computer data processing system is configured to send at least one reminder to the patient device, the reminder includes alerts for future medical conditions and recommended improvements determined by the computer data processing system.

According to one or more embodiments, A system for determining a patient health score for predicting a likelihood of a future medical condition, comprises a patient device and a computer data processing system in communication with the patient device. The computer data processing system is configured to: receive, from the patient device, a set of patient records associated with a patient, the set of patient records including at least one of medical claims records, prescription records, diet records, lifestyle records, lab result records, and mental health records; store and analyze the set of patient records; generate and assign a respective score to each of the medical claims records, prescription records, diet records, lifestyle records, lab result records, and mental health records based on the analysis of the set of patient records, each score is indicative of the likelihood of a future medical condition; generate at least one recommendation for improvement so that the patient can avoid the future medical condition; and transmitting the at least one recommendation to the patient device.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a pictorial illustration of a process for health score determination and predictive analysis according to an embodiment of the invention;

FIG. 2 is a schematic illustration of a data processing system adapted for health score determination and predictive analysis according to an embodiment of the invention;

FIG. 3 is a flow chart illustrating a process for health score determination and predictive analysis according to an embodiment of the invention; and

FIG. 4 is a flow chart illustrating a process for health care determination and delivery to a patient according to an embodiment of the invention.

### DETAILED DESCRIPTION OF THE INVENTION

Embodiments of the invention provide for health score determination and predictive analysis. A patient's health, wellness, lifestyle and diet data can be analyzed to determine a score that the patient can visualize and see how to improve or what the patient may need to work on. The patient's health score provides the patient with a precise number to see whether their overall health is good, bad or excellent and when a patient sees the number they will try to improve their score. In one or more exemplary embodiments, the health score may be indicative of a likelihood that the patient will suffer from or experience a future medical condition or other adverse condition that is detrimental for their health or well-being.

The health score will be based on a score of each of the patient's medical claims records, prescription records, diet records, lifestyle records, lab results records and mental health records. Each score and the health score, which may also be referred to as the total weighted score, may be a number out of a hundred; however, any number or scale is within the scope of this invention. The patient's medical claims records are collected and correlated with a number depending on how many chronic conditions patients have, visits as sick, visits to the emergency room, hospital visits and patients who had acute issues, etc. The patient's medical claim records are used to find out what are the underlying issues with the patient. According to one or more embodiments, and as described herein, the score referred to herein may be a numeric value.

The prescription records are collected and correlated with a score depending on how many prescriptions the patient takes, whether the prescriptions are refilled on time, and whether the patient is taking their medicine on time, etc. The diet records are collected and correlated with a score depending on how many carbohydrates, proteins, and fat (or any other macronutrient) the patient is consuming, the patient's diet and eating habits, the patient's diet restrictions, etc. to determine the patient's overall nutrition. The lifestyle records are collected and correlated with a score depending on how many steps the patient takes and hours of sleep, sleep cycle, active energy, resting heart rate, etc. of the patient to determine or indicate how active the patient is in their lifestyle. The lab results records are collected and correlated with a score depending on the patient's selected lab results that are measured against targeted ranges of acceptance. The mental health records are collected and correlated with a score depending on the patient's mental health, stress, stability, etc. The mental health score may also be assigned directly by a mental health counselor who will evaluate the patient's mental health, stress, stability, etc. to assign the score.

The medical claims records, prescription records, diet records, lifestyle records, lab results records and mental health records, as well as the associated scores, of a multiplicity of patients may be stored and analyzed with Artificial Intelligence algorithms to determine a correlation between each of the records and scores for predictive analysis of future medical conditions and recommended improvements to avoid the future medical conditions (e.g., suggesting a change in diet or exercise). The future medical conditions and reminders for recommended improvements to avoid the future medical conditions are then displayed to the patient on their own device such as, for example, a computer, laptop, tablet, cell phone, smart phone, smart watch, or other smart device. The future medical conditions may include the chances of a patient getting a heart attack, developing chronic conditions, going to urgent care, being hospitalized and going to the emergency room, etc. The future medical conditions may be limited to the next 3 to 6 months for the patient or any amount of time in the future. The future medical condition prediction will help patients understand what will be the important factor and what they need to keep in mind in order to avoid that condition.

The recommended improvements will include recommendations to improve the patient's health, such as exercises, sleep requirements, meditation requirements, prescription requirements, etc. The recommended improvements may be sent as push notifications, text messages, or any other type of audible, visual, and/or tactile notification to the patient's device(s) to remind the patient about the recommended improvements and the patient's health score. For example, the notification to the patient's smart phone will tell the patient to do one particular exercise for 3 days, and if the patient doesn't exercise, the notifications will proactively be sent to the patient to remind the patient that they haven't exercised, which will affect their health score. It is to be understood that the computer data processing system (discussed below) is configured to determine whether the patient performed the exercise through quantitative and qualitative data measures.

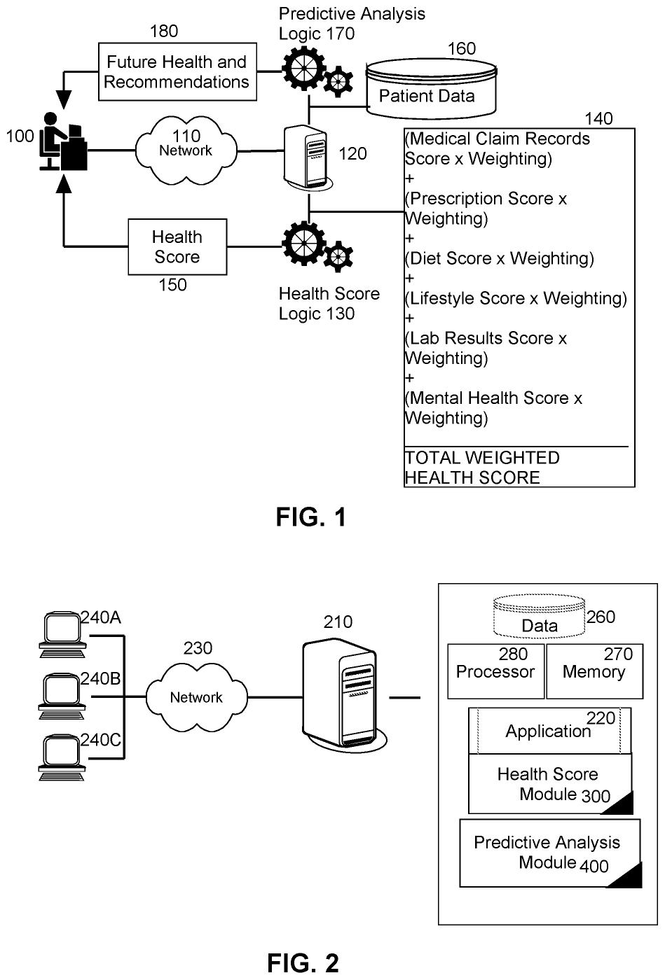

In further illustration, FIG. 1 pictorially shows a process for health score determination and predictive analysis according to an embodiment of the invention. The process may be implemented by a computer data processing system that is in communication with one or more end user or patient devices. The computer data processing system may be developed, maintained, and operated by a multidisciplinary team of one or more healthcare providers or may be integrated with the data processing system of a healthcare facility. It is to be understood that the use of the term “patient” in “patient devices” is not intended to be limiting. As such, the patient device may refer to any computing device that is used by patient (whether owned by the patient or not) to communicate with the computer data base processing system.

As shown in FIG. 1, an end user or patient 100 is connected to the server 120 over network 110 by a computing device (i.e., the patient device) which is configured to enable and facilitate communication between the patient device and server 120. The server 120 is programmed and/or configured to store the patient's medical claims records, prescription records, diet records, lifestyle records, lab results records and mental health records. As can be seen in 140, Health Score Logic 130 determines scores for each of the records, applies a weighting value to each of the scores of each of the records, and determines a total weighted health score of the patient by calculating a sum of each weighted score. The health score 150 of the patient is the total weighted health score as determined in 140. The health score 150 is then displayed to the patient on their device.

As further shown in FIG. 1, the server 120 may also store patient data 160, which includes the medical claims records, prescription records, diet records, lifestyle records, lab results records and mental health records, as well as the associated scores, of a multiplicity of patients. Predictive Analysis Logic 170 may determine Future Health and Recommendations 180 from patient data 160. The Future Health and Recommendations 180 are then displayed to the patient.

As previously mentioned, the process shown in FIG. 1 may be implemented in a computer data processing system. In further illustration, FIG. 2 schematically shows a data processing system adapted for health score determination and predictive analysis according to an embodiment of the invention. The system communicates over a network 230 with a server 210 and the system includes at least one processor 280 and memory 270 and fixed storage 260 disposed within the system. The system includes an application 220 with health score module 300 and a predictive analysis module 400. The patient computing devices 240A, 240B, 240C communicate over the network 230. Although three computing devices are shown, any amount of computing devices are contemplated by this invention. The data in fixed storage 260 also includes the medical claims records, prescription records, diet records, lifestyle records, lab results records and mental health records for each of the patients using computing devices 240A, 240B, 240C which are analyzed by Health Score Module 300 to determine each of the patient's respective health score, which is then displayed to the respective patient. The data in fixed storage 260 also includes the medical claims records, prescription records, diet records, lifestyle records, lab results records and mental health records, as well as the associated scores, of a multiplicity of patients which are analyzed by Predictive Analysis Module 400 with Artificial Intelligence algorithms to determine a correlation between each of the records and scores for a predictive analysis for future medical conditions and recommended improvements to avoid the future medical conditions which are then displayed to the respective patient.

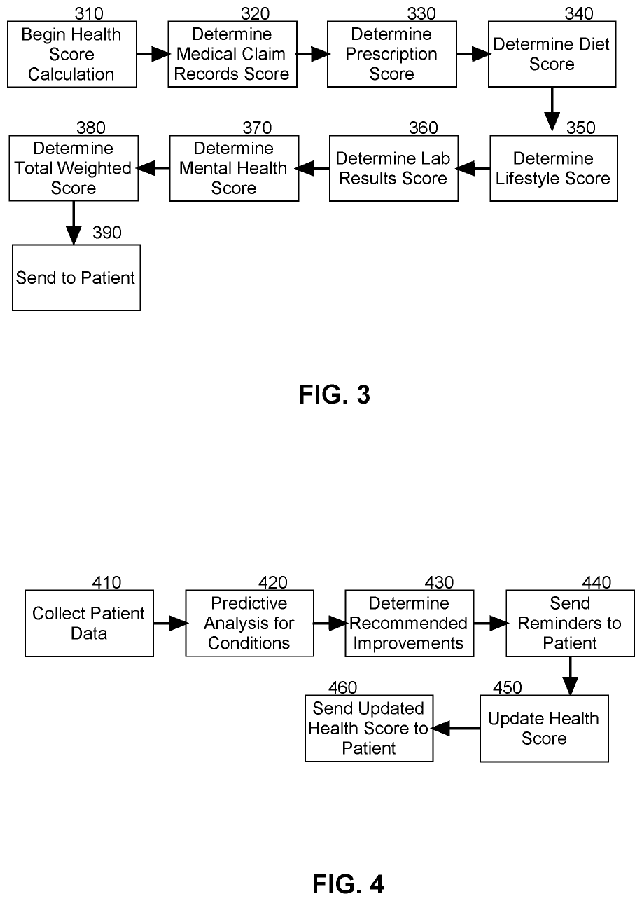

In further illustration of the operation of the module 300, FIG. 3 is a flow chart illustrating an exemplary process for health score determination according to an embodiment of the invention. Beginning in block 310, the health score calculation begins. In block 320, a medical claims records score is determined based on the patient's medical claims records. In block 330 a prescription score is determined based on the patient's prescription records. In block 340 a diet score is determined based on the patient's diet records. In block 350, a lifestyle score is determined based on the patient's lifestyle records. In block 360, a lab results score is determined based on the patient's lab results records. In block 370, a mental health score is determined based on the patient's mental health records or assigned directly by a mental health provider. In block 380, weighting for each of the scores is determined to calculate a total weighted health score of the patient. The health score is then sent to the patient in block 390. As previously mentioned, in some embodiments, the aforementioned scores may be referred to as scores that may be used to indicate the patient's likelihood of experiencing a future medical condition. In some embodiments, a score with a low numeric value may mean that the patient is at a higher risk because they have a poor medical claims history, prescription history, lifestyle history, mental health history, etc. However, in other embodiments, it is to be understood that a score with a high numeric value is associated with higher risk. In other words, the clinician or healthcare provider may preselect whether high or low numeric values for each score are indicative of higher or lower risk.

In further illustration of the operation of the module 400, FIG. 4 is a flow chart illustrating an exemplary process for predictive analysis according to an embodiment of the invention. Beginning in block 410, medical claims records, prescription records, diet records, lifestyle records, lab results records and mental health records, as well as the associated scores, of a multiplicity of patients are collected. In block 420, Artificial Intelligence algorithms determine a correlation between each of the records and scores for a predictive analysis of future medical conditions and recommended improvements to avoid the future medical conditions are also determined in block 430. Reminders regarding the future medical conditions and recommended improvements are sent to the patients in block 440. In block 450, if the patient follows the recommended improvements, the patient's health score will go up, but if the patient does not follow the recommended improvements, the patient's health score will go down. The patient is able to indicate whether they followed the recommended improvements by providing a user input to the patient device that is then transmitted, relayed, or otherwise sent to the computer data processing system. The updated health score based on whether the patient followed the recommended improvement is then sent to the patient in block 460.

According to one or more embodiments, the health score identifies associated risk levels with maladaptive behaviors to flag the patient of potential dangers of specific behaviors and recommended behavioral intervention of healthcare provider engagement. The qualitative measures used include standardized medical questions and stratified levels of medical acceptance to potential condition development based on risks identified. The results are measured as a representation of a patient's overall wellbeing and are compared to a patient's other health scores via a proprietary algorithm.

According to one or more embodiments, quantitative measure development is inclusive of wearable data collected by consenting patients. Data collected measures diet, physical activity, blood pressure, pulse, glucose, sleep patterns, breathing patterns, related to age and body mass index. These measures are stratified in acceptance levels and incorporated into health score results, including weekly updates to address deficiencies.

According to one or more embodiments, use of health scores can provide beneficial individualized health information that can reduce the occurrence of maladaptive lifestyle behaviors that are commonly associated with costly health conditions. Continued use of health score to increase healthy decision making can result in reducing overall risk of developing diseases, which can be correlated to a longer life expectancy than a patient who does not use the health score. Utilizing the health score technology allows baseline health behaviors to be quantified into a unique tangible system that provides patients an accurate representation of their current health strengths & needs in an intuitive format aimed at promoting improving their health.

According to one or more embodiments, a health score in a number format is generated by the computer data processing system for a user/patient based on the information or data provided to us with their consent. This health score utilizes various areas of the user that includes their responses to some questionnaires and also data collected from them like vitals and diet, nutrition and prescriptions etc. A questionnaire is created that includes various aspects of health of any user or patient that assess the state of health of that particular user based on some pointer scale. The questionnaire type may include one or more broad categories like mental and general health. The questionnaires developed are based on some standard formats like SF-36. The points and number of questions and types of questions used may vary depending on the needs of the algorithm and subject matter experts' opinions. The health score algorithm assigns different points based on the responses given by the user to the questionnaires given and also any smart data collected with the consent of the user. By assigning the points and also the weightage given to each question or vital data, a mathematical formula employed by the computer data processing system will help generate the final score. Here, the weightage given to questions or vital data may vary based on either the subject matter expert's decision or the weights can be generated through some correlation coefficient mathematical formula of the data collected from the user with their consent. The vital readings included here may vary. The calculation of the score depends on various factors like points system, weightage calculation from different usage, the scale used and also different sources like questionnaires, data from user, vitals, diet and nutrition, prescriptions, etc. These points, weightages, and scales can be varied according to the needs of subject matter experts and to the necessity for the score generation. The importance for the questions, vitals, and diet are given by using the data with the help of mathematical formulas like correlation coefficient. By utilizing the points, weightages, scales used here, the overall health score for the user is generated. Factors will vary in order to generate more accurate scores by utilizing the data collected in the near future.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the present invention.

Having thus described the invention of the present application in detail and by reference to embodiments thereof, it will be apparent that modifications and variations are possible without departing from the scope of the invention defined in the appended claims as follows:

What is claimed is:

1. A method of determining a patient health score for predicting a likelihood of a future medical condition, comprising the steps of:
receiving, from a patient, a set of patient records associated with the patient, the set of patient records being received by a computer data processing system maintained by an associated healthcare provider;
storing, at the computer data processing system, the set of patient records;
analyzing, at the computer data processing system, the set of patient records;
generating and assigning, at the computer data processing system, at least one score to the set of patient records based on the analysis of the records, each score being indicative of the likelihood of a future medical condition;
generating, at the computer data processing system, at least one recommendation for improvement to the patient so that the patient can avoid the future medical condition; and
transmitting the at least one recommendation from the computer data processing system to a patient device.

2. The method of claim 1, wherein the set of patient records includes at least one of medical claims records, prescription records, diet records, lifestyle records, lab result records, and mental health records.

3. The method of claim 2, wherein the medical claims records, prescription records, diet records, lifestyle records, lab result records, and mental health records are each assigned a respective score.

4. The method of claim 3, further comprising:
determining, via a health score logic of the computer data processing system, a weighting for each of the respective scores; and
applying the weighting to each respective score to calculate a weighted score.

5. The method of claim 4, further comprising:
determining a total weighted health score based on a sum of each weighted score; and
providing the total weighted health score to the patient.

6. The method of claim 1, wherein the computer data processing system has a Predictive Analysis Logic and the at least one recommendation for improvement is determined by the Predictive Analysis Logic.

7. The method of claim 1, the at least one recommendation from the computer data processing system to a patient device is transmitted via at least one of a wired and wireless connection to the patient device.

8. The method of claim 1, further comprising:
routinely updating the at least one score based on whether the patient follows the recommendation for improvement.

9. A system for determining a patient health score for predicting a likelihood of a future medical condition, comprising:
a patient device; and
a computer data processing system in communication with the patient device, the computer data processing system being configured to:
receive, from the patient device, a set of patient records associated with a patient;
store and analyze the set of patient records;
generate and assign at least one score to the set of patient records based on the analysis of the set of patient records, each score being indicative of the likelihood of a future medical condition;
generate at least one recommendation for improvement so that the patient can avoid the future medical condition; and
transmit the at least one recommendation to the patient device.

10. The system of claim 9, wherein the set of patient records includes at least one of medical claims records, prescription records, diet records, lifestyle records, lab result records, and mental health records.

11. The system of claim 10, wherein the medical claims records, prescription records, diet records, lifestyle records, lab result records, and mental health records are each assigned a respective score.

12. The system of claim 11, wherein the computer data processing system is further configured to:
determine a weighting for each of the respective scores; and
apply each weighting to each respective score to calculate a weighted score.

13. The system of claim 12, wherein the computer data processing system is further configured to:
determine a total weighted health score based on a sum of each weighted score; and
transmit the total weighted health score to the patient device.

14. The system of claim 13, wherein the computer data processing system is further configured to at least periodically update the total weighted health score based on whether the patient follows the recommended improvements.

15. The system of claim 9, wherein the computer data processing system is operated by one or more healthcare providers.

16. The system of claim 9, wherein the computer data processing system is in communication with the patient device by at least one of a wired and wireless connection.

17. The system of claim 9, wherein the computer data processing system is in communication with a plurality of patient devices.

18. The system of claim 9, wherein the computer data processing system is configured to receive a set of patient records from each patient device.

19. The system of claim 9, wherein after the computer data processing system transmits the recommendation to the patient device, the computer data processing system is configured to send at least one reminder to the patient device, the reminder includes alerts for future medical conditions and recommended improvements determined by the computer data processing system.

20. A system for determining a patient health score for predicting a likelihood of a future medical condition, comprising:
a patient device; and
a computer data processing system in communication with the patient device, the computer data processing system being configured to:
receive, from the patient device, a set of patient records associated with a patient, the set of patient records including at least one of medical claims records, prescription records, diet records, lifestyle records, lab result records, and mental health records;
store and analyze the set of patient records;
generate and assign a respective score to each of the medical claims records, prescription records, diet records, lifestyle records, lab result records, and mental health records based on the analysis of the set of patient records, each score being indicative of the likelihood of a future medical condition;
generate at least one recommendation for improvement so that the patient can avoid the future medical condition; and
transmitting the at least one recommendation to the patient device.
